(19) United States
(12) Patent Application Publication
(10) Pub. No.: US 2015/0313275 A1
(43) Pub. Date: Nov. 5, 2015

(54) LIQUID AEROSOL FORMULATION OF AN ELECTRONIC SMOKING ARTICLE

(71) Applicant: Altria Client Services, Inc., Richmond, VA (US)

(72) Inventors: Adam N. ANDERSON, Moseley, VA (US); Pauline MARCQ, Richmond, VA (US); Gerd KOBAL, Sandy Hook, VA (US); Mark RUSYNIAK, Richmond, VA (US); Kent B. KOLLER, Chesterfield, VA (US); San LI, Midlothian, VA (US); Yevgeniya V. PREPELITSKAYA, Henrico, VA (US); Niti H. SHAH, Henrico, VA (US)

(21) Appl. No.: 14/602,099

(22) Filed: Jan. 21, 2015

Related U.S. Application Data
(60) Provisional application No. 62/029,222, filed on Jul. 25, 2014.
(60) Provisional application No. 61/986,536, filed on Apr. 30, 2014.

(51) Int. Cl. A24B 15/10; A24F 47/00; A61K 47/12; A61K 31/465; A61K 9/12

(52) U.S. Cl. CPC A24B 15/10; A61K 31/465; A61K 9/12; A61K 47/12; A24F 47/00

(57) ABSTRACT

A liquid aerosol formulation for an electronic smoking article includes an aerosol former, optionally water, nicotine, and an acid. The acid can include one or more of pyruvic acid, formic acid, oxalic acid, glycolic acid, acetic acid, isovaleric acid, valeric acid, propionic acid, octanoic acid, lactic acid, levulinic acid, sorbic acid, malic acid, tartaric acid, succinic acid, citric acid, benzoic acid, oleic acid, aconitic acid, butyric acid, cinnamic acid, decanoic acid, 3,7-diemthyl-6-octenoic acid, 1-glutamic acid, heptanoic acid, hexanoic acid, 3-hexenoic acid, trans-2-hexenoic acid, isobutyric acid, lauric acid, 2-methylbutyric acid, 2-methylvaleric acid, myristic acid, nonanoic acid, palmitic acid, 4-pentenoic acid, phenylacetic acid, 3-phenylpropionic acid, hydrochloric acid, phosphoric acid and sulfuric acid.

The current application claims priority from U.S. Provisional Patent Application No. 61/986,536, filed on Apr. 30, 2014 and titled “Liquid Aerosol Formulation of an Electronic Smoking Article,” and from U.S. Provisional Patent Application No. 62/029,222, filed on Jul. 25, 2014 and titled “Liquid Aerosol Formulation of an Electronic Smoking Article,” both of these applications being incorporated herein in their entirety by reference.

### BACKGROUND OF THE INVENTION

1. Field of the Invention

Example embodiments relate generally to a liquid aerosol formulation for e-vaping devices.

2. Related Art

Electronic vaping devices are used to vaporize a liquid material into an aerosol or “vapor” in order for an adult vaper to inhale the vapor. These electronic vaping devices may be referred to as e-vaping devices. E-vaping devices include a heater which vaporizes liquid material to produce an aerosol. An e-vaping device may include several e-vaping elements including a power source, a cartridge or e-vaping tank including the heater and along with a reservoir capable of holding the liquid material.

A lit-end smoking article produces an aerosol known to create pleasant sensory experience for adult smokers, including a low to moderate harshness response in the throat and a perceived warmth in the chest. The preferred levels of harshness in the throat and perceived warmth in the chest will differ amongst adult cigarette smokers.

### SUMMARY OF THE INVENTION

At least one example embodiment relates to an e-vaping device.

In one example embodiment, the e-vaping includes a first section. The first section includes an outer cylindrical tube extending in a longitudinal direction, an inner cylindrical tube within the outer cylindrical tube, and a liquid supply comprising a liquid material. The liquid supply is contained in an outer annulus between the outer cylindrical tube and the inner cylindrical tube. The first section further includes a heater located in the inner cylindrical tube, a wick in communication with the liquid supply and in communication with the heater, a mouth piece in fluid communication the inner cylindrical tube at a proximal end of the first section, and a male threaded connector at a distal end of the first section. The male threaded connector has first threads with a non-standard pitch. The e-vapor device further includes a second section. The second section includes a power supply, and a female threaded connector at a proximal end of the second section. The female threaded connector has second threads mating with the non-standard pitch of the first threads.

At least one example embodiment relates to an electronic cigarette or e-vaping device that is configured to provide a pleasant sensory experience for adult smokers that is similar to the sensory experience enjoyed while smoking a lit end cigarette.

Another example embodiment relates to an electronic cigarette or e-vaping device that is configured to provide a sensory experience including levels of harshness in the throat and perceived warmth in the chest that are similar to those experienced by adult smokers when smoking a lit end cigarette.

In one embodiment, a liquid aerosol formulation of an electronic smoking article or e-vaping device includes a mixture of an aerosol former, optionally water, nicotine and an acid having a boiling point of at least about 100° C. and configured to volatilize when heated by a heater in the electronic smoking article. The liquid aerosol formulation is configured to form an aerosol having a particulate phase and a gas phase when heated by the heater in the electronic smoking article, the particulate phase contains protonated nicotine and the gas phase contains unprotonated nicotine, and the aerosol has a majority amount of the protonated nicotine and a minority amount of the unprotonated nicotine.

In one embodiment, a liquid aerosol formulation of an electronic smoking article includes a mixture of an aerosol former, optionally water, nicotine and an acid. The liquid aerosol formulation is configured to form an aerosol having a gas phase upon operation of the electronic smoking article. The acid is operative upon the aerosol so as to reduce an amount of nicotine content in the gas phase of the aerosol in comparison to the aerosol being formed in the absence of the acid.

In one embodiment, a liquid aerosol formulation of an electronic smoking article includes a mixture of an aerosol former, optionally water, nicotine in an amount of 2% by weight or greater of a total weight of the formulation, and an acid. The liquid aerosol formulation is configured to form an aerosol upon operation of the electronic smoking article. The acid is operative upon the aerosol so as to reduce the amount of perceived throat harshness in comparison to the aerosol formed in the absence of the acid.

In one embodiment, an electronic smoking article configured to produce an aerosol includes a liquid reservoir containing a liquid aerosol formulation, a heater and an arrangement to communicate the reservoir with the heater. The heater is configured to vaporize the liquid aerosol formulation. The liquid aerosol formulation is configured to form an aerosol having a nicotine gas phase component upon operation of the electronic smoking article. The liquid aerosol formulation includes a mixture of an aerosol former, optionally water, nicotine and an acid. The acid is included in an amount sufficient to reduce the nicotine gas phase component by about 70% by weight or greater of a nicotine gas phase component produced upon operation of the electronic smoking article with the formula but without the acid. In other embodiments, it may be sufficient to reduce the gas phase nicotine content in the range of about 40 to about 70% by weight with the addition of an acid.

In one embodiment, an electronic smoking article configured to produce an aerosol includes a liquid reservoir containing a liquid aerosol formulation, a heater and an arrangement to communicate the reservoir with the heater. The heater is configured to vaporize the liquid aerosol formulation. The liquid aerosol formulation is configured to form an aerosol upon operation of the electronic smoking article. The liquid aerosol formulation includes a mixture of an aerosol former, optionally water, nicotine in an amount of 2% by weight or greater of the total weight of the liquid aerosol formulation, and an acid. The acid is operative upon the aerosol so as to reduce the amount of perceived throat harshness by a user in comparison to the aerosol being formed upon operation of the electronic smoking article without the acid.

In one embodiment, a liquid aerosol formulation of an electronic smoking article includes a mixture of an aerosol former, optionally water, nicotine and an acid. The liquid aerosol formulation is configured to form an aerosol upon operation of the electronic smoking article. The acid is selected to have a liquid to aerosol transfer efficiency of about 50% or greater and in an amount sufficient to reduce the nicotine gas phase component by about 70% by weight or greater of a nicotine gas phase component produced upon operation of the electronic smoking article with the formula without the acid.

In one embodiment, a method of reducing a gas phase nicotine content of an aerosol generated in an electronic smoking article includes: (i) obtaining a liquid aerosol formulation including a mixture of an aerosol former, optionally water, and nicotine; (ii) operating the electronic smoking article to vaporize the liquid aerosol formulation to form the aerosol having a nicotine gas phase component; and (iii) prior to the vaporizing, including an acid in the aerosol formulation in an amount sufficient to reduce the nicotine gas phase component by about 70% by weight or greater of a nicotine gas phase component produced upon operation of the electronic smoking article with the formula without the acid.

In one embodiment, a method of reducing perceived throat harshness of an aerosolized formulation of an electronic smoking article including nicotine includes adding an acid to the formulation in an amount to reduce an amount of perceived throat harshness in comparison to the aerosolized formulation being formed without the acid.

In one embodiment, a method of reducing perceived throat harshness of an aerosolized formulation of an electronic smoking article including nicotine includes reducing a nicotine gas phase component of the aerosolized formulation by adding an acid to the formulation.

In one embodiment, a liquid aerosol formulation of an electronic smoking article includes an aerosol former, nicotine, and an acidic compound, wherein the liquid aerosol formulation is configured to form an aerosol having a particulate phase and a gas phase when heated in the electronic smoking article, and wherein a concentration of nicotine in the gas phase is equal to or smaller than substantially 1% by weight of the total nicotine delivered. As a result, 99% of the total nicotine delivered in the aerosol is in the particulate phase. In one embodiment, a concentration of nicotine in the gas phase is equal to or smaller than substantially 1% by weight of the aerosolized nicotine of the total nicotine delivered.

In one embodiment, the acidic compound includes at least one of pyruvic acid, formic acid, oxalic acid, glycolic acid, acetic acid, isovaleric acid, valeric acid, propionic acid, octanoic acid, lactic acid, sorbic acid, malic acid, tartaric acid, succinic acid, citric acid, benzoic acid, oleic acid, aconitic acid, butyric acid, cinnamic acid, decanoic acid, 3,7-dimethyl-6-octenoic acid, 1-glutamic acid, heptanoic acid, hexanoic acid, 3-hexenoic acid, trans-2-hexenoic acid, isobutyric acid, lauric acid, 2-methylbutyric acid, 2-methylvaleric acid, myristic acid, nonanoic acid, palmitic acid, 4-pentenoic acid, phenylacetic acid, 3-phenylpropionic acid, hydrochloric acid, phosphoric acid and sulfuric acid.

In one embodiment, the acidic compound includes pyruvic acid, lactic acid, benzoic acid and acetic acid.

In one embodiment, the concentration of the nicotine is between substantially 1.5% by weight and substantially 6% by weight. The concentration of the nicotine may also be between substantially 3% by weight and substantially 4.5% by weight.

In one embodiment, the concentration of the acidic compound is between substantially 0.25% by weight and substantially 2% by weight. The concentration of the acidic compound may also be between substantially 0.5% by weight and substantially 1.5% by weight, or between substantially 1.5% by weight and substantially 2% by weight. The acidic compound may include between 2 and 10 acids. For example, the acidic compound may include 4 acids. The acidic compound may also include substantially equal parts of each individual acid included in the compound. For example, the acidic compound may include substantially equal parts of pyruvic acid, lactic acid, benzoic acid and acetic acid. Alternatively, the acidic compound may include more of the pyruvic acid than one or more of the lactic acid, the benzoic acid and the acetic acid.

In one embodiment, the acidic compound includes at least one of pyruvic acid, acetic acid, isovaleric acid, valeric acid, propionic acid, octanoic acid, lactic acid, sorbic acid, malic acid, tartaric acid, succinic acid, citric acid, benzoic acid, oleic acid, aconitic acid, butyric acid, cinnamic acid, decanoic acid, 3,7-dimethyl-6-octenoic acid, 1-glutamic acid, heptanoic acid, hexanoic acid, 3-hexenoic acid, trans-2-hexenoic acid, isobutyric acid, lauric acid, 2-methylbutyric acid, 2-methylvaleric acid, myristic acid, nonanoic acid, palmitic acid, 4-pentenoic acid, phenylacetic acid, 3-phenylpropionic acid, phosphoric acid and sulfuric acid.

In one embodiment, the liquid aerosol formulation includes substantially 3% nicotine by weight, substantially 0.5% pyruvic acid, substantially 0.4% lactic acid and 0.1% benzoic acid.

In one embodiment, the liquid aerosol formulation includes substantially 3% nicotine by weight, substantially 0.5% pyruvic acid, substantially 0.4% lactic acid and substantially 0.1% hydrochloric acid.

In one embodiment, the liquid aerosol formulation includes substantially 3% nicotine by weight, substantially 0.5% nicotine bitartrate and substantially 0.2% hydrochloric acid.

In one embodiment, the liquid aerosol formulation includes substantially 3% nicotine by weight, substantially 0.5% pyruvic acid, substantially 0.4% lactic acid and substantially 0.5% tartaric acid.

In one embodiment, the liquid aerosol formulation includes substantially 3% nicotine by weight, substantially 0.5% pyruvic acid, substantially 0.4% lactic acid, substantially 0.1% benzoic acid and substantially 0.1% oleic acid.

In one embodiment, the liquid aerosol formulation includes substantially 3% nicotine by weight, substantially 0.5% nicotine bitartrate, substantially 0.5% pyruvic acid, substantially 0.4% lactic acid and substantially 0.1% benzoic acid.

In one embodiment, the liquid aerosol formulation includes substantially 3% nicotine by weight, substantially 0.5% nicotine bitartrate, substantially 0.5% pyruvic acid, substantially 0.4% lactic acid, substantially 0.1% benzoic acid and substantially 1% oleic acid.

In one embodiment, the liquid aerosol formulation includes substantially 3% to 5% nicotine by weight, substantially 0.036% to 0.144% formic acid, substantially 0.35% to 1.4% pyruvic acid, substantially 0.013% to 0.052% oxalic acid and substantially 0.05% to 0.2% glycolic acid. For example, the liquid aerosol formulation includes substantially 3% nicotine by weight, substantially 0.036% formic acid, substantially 0.35% pyruvic acid, substantially 0.013% oxalic acid and substantially 0.05% glycolic acid.

In one embodiment, the liquid aerosol formulation includes substantially 3% to 5% nicotine by weight, substantially 1% to 2% nicotine bitartrate and substantially 0.4% to 0.9% acetic acid. For example, the liquid aerosol formulation includes substantially 3% nicotine by weight, substantially 1% nicotine bitartrate and substantially 0.4% acetic acid.

In one embodiment, the liquid aerosol formulation includes substantially 3% to 5% nicotine by weight, substantially 0.5% to 1% nicotine bitartrate and substantially 0.5% to 1% of a mixture of substantially equal parts pyruvic acid, benzoic acid, lactic acid and acetic acid.

In one embodiment, the liquid aerosol formulation includes substantially 3% to 5% nicotine by weight, substantially 0.5% to 1% nicotine bitartrate and substantially 0.01% to 0.1% hydrochloric acid.

In one embodiment, the liquid aerosol formulation includes substantially 3% to 5% nicotine by weight, substantially 0.5% to 1% oleic acid and substantially 0.5% to 1.5% of a mixture of substantially equal parts pyruvic acid, benzoic acid, lactic acid and acetic acid.

In one embodiment, the liquid aerosol formulation includes substantially 3% to 5% nicotine by weight, substantially 0.1% to 0.5% sorbic acid, substantially 0.1% to 0.5% tartaric acid and substantially 0.1% to 0.75% pyruvic acid. For example, when the concentration of nicotine by weight is substantially 5%, hydrochloric acid may be added at a concentration of substantially 0.01% to 0.1%.

In one embodiment, the liquid aerosol formulation includes substantially 3% to 5% nicotine by weight, substantially 0.1% to 0.5% succinic acid, substantially 0.1% to 0.75% pyruvic acid and substantially 0.1% to 0.5% lactic acid. For example, when the concentration of nicotine by weight is substantially 5%, hydrochloric acid may be added at a concentration of substantially 0.01% to 0.1%, or tartaric acid may be added at a concentration of substantially 0.1% to 0.25%.

In one embodiment, a liquid aerosol formulation includes a combination of benzoic acid, lactic acid, acetic acid, and pyruvic acid. The benzoic acid, the lactic acid, the acetic acid and the pyruvic acid may be in equal proportions. In addition, the resulting aerosol may include an amount of nicotine in the gas phase of less than or equal to substantially 1% of the total nicotine delivered. As a result, 99% of the total nicotine delivered in the aerosol is in the particulate phase. The above combination of the benzoic acid, the lactic acid, the acetic acid and the pyruvic acid, together with the nicotine concentration in the gas phase of the aerosol of equal to or less than substantially 1% of the total nicotine delivered, results in an inhalable aerosol that has an improved combination of harshness and warmth in chest compared to other combinations of acids and higher concentrations of nicotine in the gas phase.

### BRIEF DESCRIPTION OF THE DRAWINGS

The above and other features and advantages of example embodiments will become more apparent by describing in detail, example embodiments with reference to the attached drawings. The accompanying drawings are intended to depict example embodiments and should not be interpreted to limit the intended scope of the claims. The accompanying drawings are not to be considered as drawn to scale unless explicitly noted.

FIG. 1 is a side view of an electronic smoking article, according to an example embodiment;

FIG. 2 is a cross-sectional view of an electronic smoking article, according to an example embodiment;

FIG. 3 is a cross-sectional view of another example embodiment of an electronic smoking article;

FIG. 4 is a cross-sectional view of an electronic smoking article according to an example embodiment;

FIG. 5 is a graphical representation of gas phase nicotine contents by puff-by-puff analysis of a control formulation, a nicotine bitartrate (NB)-containing liquid aerosol formulation, and a liquid aerosol formulation containing formic acid, pyruvic acid, oxalic acid, and glycolic acid, according to an example embodiment;

FIG. 6 is a graphical representation of residue formation of a control formulation, a nicotine bitartrate-containing liquid aerosol formulation, and a liquid aerosol formulation containing formic acid, pyruvic acid, oxalic acid, and glycolic acid, according to an example embodiment; and

FIG. 7 is a graphical representation of aerosol (particulate phase) mass delivery of a control formulation, a nicotine bitartrate-containing liquid aerosol formulation, and a liquid aerosol formulation containing formic acid, pyruvic acid, oxalic acid, and glycolic acid, according to an example embodiment.

### DETAILED DESCRIPTION

Some detailed example embodiments are disclosed herein. However, specific structural and functional details disclosed herein are merely representative for purposes of describing example embodiments. Example embodiments may, however, be embodied in many alternate forms and should not be construed as limited to only the embodiments set forth herein.

Accordingly, while example embodiments are capable of various modifications and alternative forms, embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It should be understood that, although the terms first, second, third, etc. may be used herein to describe various elements, components, regions, layers and/or sections, these elements, components, regions, layers, and/or sections should not be limited by these terms. These terms are only used to distinguish one element, component, region, layer, or section from another region, layer, or section. Thus, a first element, component, region, layer, or section discussed below could be termed a second element, component, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

Example embodiments are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments should not be construed as limited to the shapes of regions illustrated herein but are to include deviations in shapes that result, for example, from manufacturing. Thus, the regions illustrated in the figures are schematic in nature and their shapes are not intended to illustrate the actual shape of a region of a device and are not intended to limit the scope of example embodiments.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

In one embodiment, an electronic smoking article includes a liquid supply reservoir containing a liquid aerosol formulation. The liquid aerosol formulation is delivered to a heater of the electronic smoking article where the liquid aerosol formulation is heated and volatilized to form an aerosol upon operation of the electronic smoking article. In an example embodiment, the liquid aerosol formulation includes a mixture of molecular nicotine (unprotonated and uncharged) and an acid, which protonates nearly all of the molecular nicotine in the liquid formulation, so that upon heating of the liquid aerosol formulation by a heater in the electronic smoking article, an aerosol having a majority amount of protonated nicotine and a minority amount of unprotonated nicotine is produced, whereby only a minor portion of all the volatilized (vaporized) nicotine typically remains in the gas phase of the aerosol. The fraction of nicotine in the gas phase may contribute to perceptions of throat harshness and/or other perceived off-tastes. Reducing the proportional level of nicotine in the gas phase may improve the perceived subjective deficits associated with nicotine in the gas phase. For example, a proportion of nicotine in the gas phase of the aerosolized nicotine may be substantially 1% of the total nicotine delivered.

According to at least one example embodiment, the acid: (a) readily protonates nicotine and thus reduces production of gas phase nicotine; (b) does not impede operation of a wick and a heater of an electronic cigarette or e-vaping device; (c) does not degrade production levels of “per puff” aerosol mass; (d) has minimal sensory impact and/or can be included at levels wherein its sensory impact becomes undetectable or unobjectionable; (e) is sufficiently, thermally stable to withstand a heating cycle of an electronic smoking article so that at least a substantial portion of the acid enters the gas vapor phase as an acid; (f) volatilizes (or vaporizes) at the operation temperature of the heater of an electronic smoking article; and/or (g) is condensable at ambient temperatures. In general, a relatively strong acid has a low pKa value and can readily protonate nicotine. In one embodiment, the acid has an acceptably low tendency to produce a char and/or a polymeric residue upon operation of the electronic smoking article and thus does not impede the capillarity of the wick.

According to at least one example embodiment, the acid has the capacity to transfer into an aerosolized formulation. Transfer efficiency of an acid is the ratio of the mass fraction of the acid in the aerosolized formulation to the mass fraction of the acid in the liquid formulation, as measured by known techniques such as, e.g., ion chromatography. In one embodiment, the acid has a liquid to aerosol transfer efficiency of about 50% or greater, more preferably about 60% or greater. For example, pyruvic acid, lactic acid, oxalic acid, acetic acid and glycolic acid have aerosol transfer efficiency of about 50% or greater. In one embodiment, the liquid aerosol formulation includes an acid having an aerosol transfer efficiency of about 50% or greater. In another embodiment, the liquid aerosol formulation excludes (would not include or be exclusive of) any acid having aerosol transfer efficiency of about 25% or less. Moreover, the acid is preferably classified as generally recognized as safe (GRAS).

In another embodiment, the acid is added within an acceptable sensorial amount according to a sensory impact associated with the acid. For example, to some adult consumers, acetic acid, when added at certain levels, may impart a “vinegar” sensorial response. Accordingly, in one embodiment, the acetic acid content may be limited to levels below where such sensory impact arises. Other acids can also be used in combination with the acetic (or other) acid in a similar manner so as to establish an acid complex wherein the desired level of acid functionality is achieved (with multiple acids), but with each acid being included at a level below where noticeable or objectionable sensory impact may arise.

According to at least one example embodiment, the acid has a boiling point of at least about 100° C., and may be included in the liquid aerosol formulation in an amount sufficient to adjust the pH of the liquid aerosol formulation in the range of about 3 to about 8.

In one embodiment, the acid is operative upon the aerosol generated from the liquid aerosol formulation upon operation of the electronic smoking article so as to reduce the amount of the nicotine content in the gas phase of the aerosol in comparison to the aerosol being formed in the absence of the acid. In one embodiment, the acid is included in an amount sufficient to reduce the amount of nicotine gas phase component by about 30% by weight or greater, preferably about 60% to about 70% by weight, more preferably, about 70% by weight or greater, and most preferably about 85% by weight or greater, of the level of nicotine gas phase component produced without the acid.

In one embodiment, the acid is operative upon the aerosol generated from the liquid aerosol formulation upon operation of the electronic smoking article so as to reduce the amount of perceived throat harshness in comparison to the aerosol formed without the acid.

According to at least one example embodiment, the liquid aerosol formulation includes one or more of pyruvic acid, formic acid, oxalic acid, glycolic acid, acetic acid, isovaleric acid, valeric acid, propionic acid, octanoic acid, lactic acid, levulinic acid, sorbic acid, malic acid, tartaric acid, succinic acid, citric acid, benzoic acid, oleic acid, aconitic acid, butyric acid, cinnamic acid, decanoic acid, 3,7-dimethyl-6-octenoic acid, 1-glutamic acid, heptanoic acid, hexanoic acid, 3-hexenoic acid, trans-2-hexenoic acid, isobutyric acid, lauric acid, 2-methylbutyric acid, 2-methylvaleric acid, myristic acid, nonanoic acid, palmitic acid, 4-pentenoic acid, phenylacetic acid, 3-phenylpropionic acid, hydrochloric acid, phosphoric acid, sulfuric acid, and combinations thereof. The acid also may be incorporated in the form of a salt. The liquid aerosol formulation also includes an aerosol former, optionally water, and optionally flavorants.

The acids may protonate nearly all of the molecular nicotine in the liquid aerosol formulation, so that upon heating of the liquid aerosol formulation in the electronic smoking article, an aerosol having a majority amount of protonated nicotine and a minority amount of unprotonated nicotine is produced, whereby only a minor portion of all the volatilized (vaporized) nicotine remains in the gas phase of the aerosol. Since the majority of the nicotine in the gas phase is protonated, the aerosol contributes a sensory response of low to mild harshness in the throat, even at elevated nicotine levels in the liquid aerosol formulation. It is believed that much of the foregoing occurs, because protonated nicotine is charged and does not enter (nor can remain in) a gas phase of the aerosol, but is instead found in a particulate phase of the aerosol. The acids in the liquid aerosol formulation may lower the pH of the aerosol formed so as to bind the nicotine in the particulate phase of the aerosol, which reduces gas phase nicotine, and accordingly reduces perceptions of throat harshness upon inhalation, and sensory off-notes perceived by some adult smokers.

Nicotine bitartrate has been included in the liquid aerosol formulation, as described in provisional patent application Ser. No. 61/856,286 to Peter Lipowicz et al., titled “LIQUID AEROSOL FORMULATION OF AN ELECTRONIC SMOKING ARTICLE” and filed Jul. 19, 2013. However, nicotine bitartrate formulations tend to deposit carbonaceous char and/or polymeric residues on and/or in the heater and wick of the electronic cigarette under certain circumstances. The use of a liquid aerosol formulation as described herein achieves the desired effects associated with use of nicotine bitartrate, but with significantly less formation of carbonaceous char and/or polymeric residues on and/or in the heater and wick.

In one embodiment, the liquid aerosol formulation is substantially nicotine bitartrate-free.

In another embodiment, the liquid aerosol formulation is substantially propellant-free.

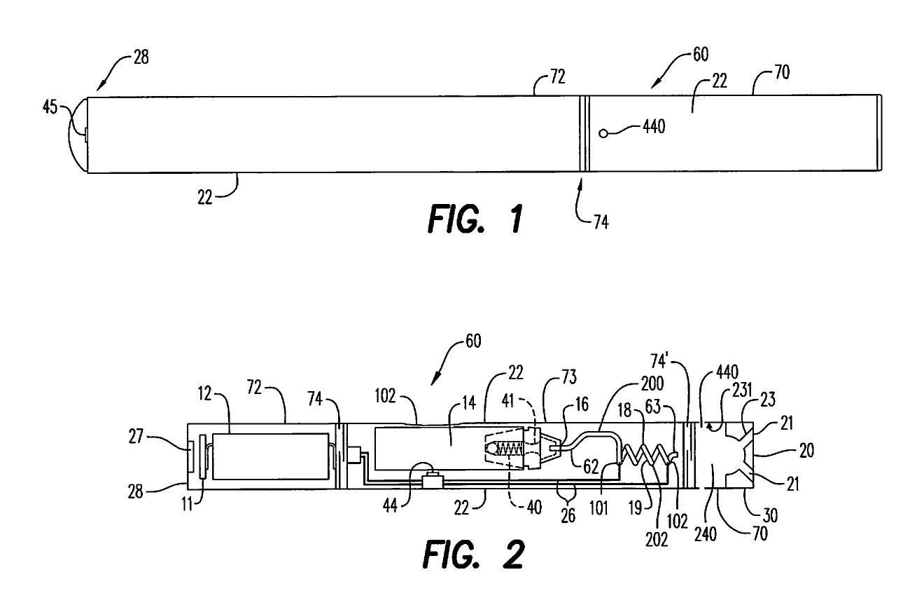

FIG. 1 is a side view of an electronic smoking article, according to an example embodiment. In FIG. 1, the liquid aerosol formulation forms an aerosol when vaporized in an electronic smoking article 60 such as, e.g., an e-vaping device, as shown in FIG. 1. The electronic smoking article 60 comprises a replaceable cartridge (or first section) 70 and a reusable fixture (or second section) 72, which are coupled together at a threaded joint 74 or by other connecting structure such as a snug-fit, snap-fit, detent, clamp and/or clasp or the like.

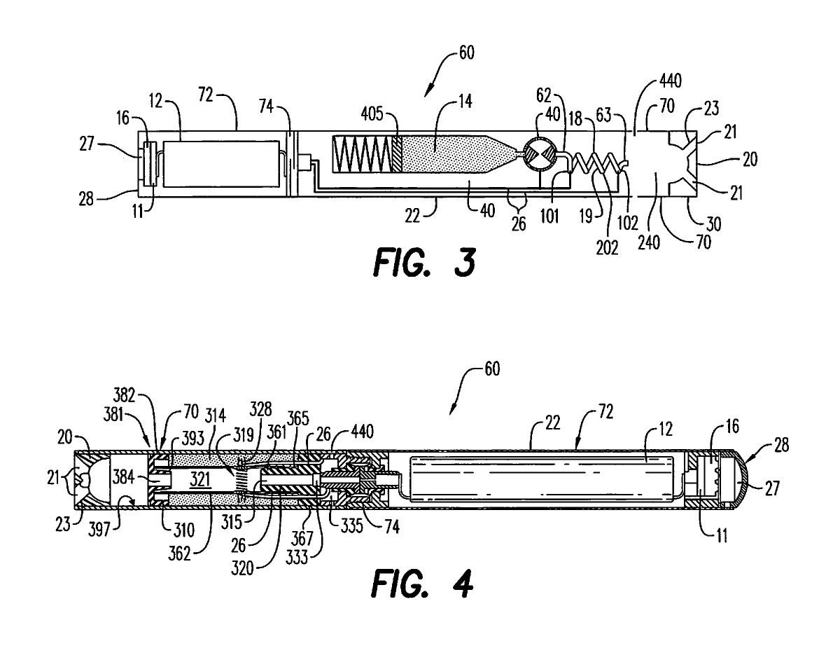

FIG. 3 is a cross-sectional view of another example embodiment of an electronic smoking article, according to an example embodiment. As shown in FIG. 3, the first section 70 can house a mouth-end insert 20, a capillary aerosol generator including a capillary tube 18, a heater 19 to heat at least a portion of the capillary tube 18, a liquid supply reservoir 14, and optionally a valve 40. Alternatively, as shown in FIG. 4, the first section 70 can house a mouth end insert 20, a heater 319, a flexible, filamentary wick 328 and a liquid supply reservoir 314 as discussed in further detail below.

The second section 72 can house a power supply 12 (shown in FIGS. 2, 3 and 4), a control circuitry 11, and optionally a puff sensor 16 (shown in FIGS. 2 and 4). The threaded portion 74 of the second section 72 can be connected to a battery charger, when not connected to the first section 70, to charge the battery or power supply 12.

FIG. 2 is a cross-sectional view of an electronic smoking article according to an example embodiment. As shown in FIG. 2, the electronic smoking article 60 can also include a middle section (third section) 73, which can house the liquid supply reservoir 14, the heater 19 and the valve 40. The middle section 73 can be configured to be fitted with a threaded joint 74′ at an upstream end of the first section 70 and a threaded joint 74 at a downstream end of the second section 72. In this example embodiment, the first section 70 houses the mouth-end insert 20, while the second section 72 houses the power supply 12 and the control circuitry 11.

In one embodiment, the first section 70, the second section 72 and the optional third section 73 include an outer cylindrical housing 22 extending in a longitudinal direction along the length of the electronic smoking article 60. Moreover, in one embodiment, the middle section 73 is disposable and the first section 70 and/or second section 72 are reusable. The sections 70, 72, 73 can be attached by threaded connections or connectors whereby the middle section 73 can be replaced when the liquid supply reservoir 14 is used up. In another embodiment, the first section 70 can also be replaceable so as to avoid the need for cleaning the capillary tube 18 and/or heater 19.

In one embodiment, the first section 70 and the second section 72 may be integrally formed without threaded connections to form a disposable electronic smoking article.

As shown in FIG. 2, the outer cylindrical housing 22 can include a cutout or depression 102 which allows a smoker to manually apply pressure to the liquid supply reservoir 14. In one embodiment, the outer cylindrical housing 22 is flexible and/or compressible along the length thereof and fully or partially covers the liquid supply reservoir 14. The cutout or depression 102 can extend partially about the circumference of the outer cylindrical housing 22. Thus, the outer cylindrical housing 22 can be formed of or include a variety of materials including plastics, rubber and combinations thereof. In one embodiment, the outer cylindrical housing 22 is formed of or includes silicone. The outer cylindrical housing 22 can be any suitable color and/or can include graphics or other indicia printed thereon. Moreover, the liquid supply reservoir 14 is compressible such that when pressure is applied to the liquid supply reservoir, liquid is pumped from the liquid supply reservoir 14 to the capillary tube 18. A pressure activated switch 44 can be positioned beneath the liquid supply reservoir 14. When pressure is applied to the liquid supply reservoir 14 to pump liquid, the switch is also pressed and a heater 19 is activated. The heater 19 can be a portion of the capillary tube 18. By applying manual pressure to the pressure switch, the power supply 12 is activated and an electric current heats the liquid in the capillary tube 18 via electrical contacts so as to volatilize the liquid.

In the example embodiment illustrated in FIG. 2, the liquid supply reservoir 14 is a tubular, elongated body formed of or including an elastomeric material so as to be flexible and/or compressible when squeezed. In one embodiment, the elastomeric material can be one of silicone, plastic, rubber, latex, and combinations thereof.

In one embodiment, the compressible liquid supply reservoir 14 has an outlet 17 in fluid communication with a capillary tube 18 so that when squeezed, the liquid supply reservoir 14 can deliver a volume of liquid material to the capillary tube 18. Contemporaneously to delivering liquid to the capillary, the power supply 12 is activated upon the application of the manual pressure on the pressure switch, and the capillary tube 18 is heated to form a heated section wherein the liquid material is volatilized. Upon discharge from the heated capillary tube 18, the volatilized material expands, mixes with air and forms an aerosol.

In one embodiment, the liquid supply reservoir 14 extends longitudinally within the outer cylindrical housing 22 of the first section 70 (shown in FIGS. 3 and 4) or the middle section 73 (shown in FIG. 2). Moreover, the liquid supply reservoir 14 contains a liquid aerosol formulation that is configured to be volatilized when heated and to form an aerosol when discharged from the capillary tube 18.

In the example embodiments illustrated in FIGS. 2 and 3, the capillary tube 18 includes an inlet end 62 in fluid communication with the outlet 17 of the liquid supply reservoir 14, and an outlet end 63 configured to expel volatilized liquid material from the capillary tube 18. In one embodiment, as shown in FIGS. 2 and 3, the liquid supply reservoir 14 may include a valve 40.

As shown in FIG. 2, the valve 40 can be a check valve configured to maintain the liquid material within the liquid supply reservoir and to open when the liquid supply reservoir 14 is squeezed and pressure is applied to the reservoir 14. In one embodiment, the check valve 40 opens when a critical, minimum pressure is reached so as to avoid inadvertent dispensing of liquid material from the liquid supply reservoir 14 or activating the heater 19. In one embodiment, the critical pressure needed to open the check valve 40 is essentially equal to or slightly less than the pressure required to press a pressure switch 44 to activate the heater 19. In one embodiment, the pressure required to press the pressure switch 44 is high enough such that accidental heating is avoided. Such arrangement avoids activation of the heater 19 in the absence of liquid being pumped through the capillary.

Advantageously, the use of a check valve 40 aids in limiting the amount of liquid that is drawn back from the capillary tube upon release of pressure upon the liquid supply reservoir 14 (and/or the switch 44) if manually pumped so as to avoid air uptake into the liquid supply reservoir 14. Presence of air degrades pumping performance of the liquid supply reservoir 14 and can degrade the liquid aerosol formulation.

Once pressure upon the liquid supply reservoir 14 is relieved, the valve 40 closes. The heated capillary tube 18 discharges liquid remaining downstream of the valve 40.

Optionally, a critical flow orifice 41 is located downstream of the check valve 40 to establish a maximum flow rate of liquid to the capillary tube 18.

As shown in FIG. 3, in other example embodiments, the valve 40 can be a two-way valve and the liquid supply reservoir 14 can be pressurized. For example, the liquid supply reservoir 14 can be pressurized using a pressurization arrangement 405 configured to apply constant pressure to the liquid supply reservoir 14. For example, pressure can be applied to the liquid supply reservoir 14 using an internal or external spring and plate arrangement which constantly applies pressure to the liquid supply reservoir 14. Alternatively, the liquid supply reservoir 14 can be compressible and positioned between two plates that are connected by springs or the liquid supply reservoir 14 could be compressible and positioned between the outer housing and a plate that are connected by a spring so that the plate applies pressure to the liquid supply reservoir 14.

In one embodiment, the capillary tube 18 of FIGS. 2 and 3 has an internal diameter of 0.01 to 10 mm, preferably 0.05 to 1 mm, and more preferably 0.05 to 0.4 mm. Capillary tubes of smaller diameter provide more efficient heat transfer to the fluid because, with the shorter distance to the center of the fluid, less energy and time is required to vaporize the liquid.

In one embodiment, the capillary tube 18 may have a length of about 5 mm to about 72 mm, more preferably about 10 mm to about 60 mm or about 20 mm to about 50 mm. In one embodiment, the capillary tube 18 is substantially straight. In other embodiments, the capillary tube 18 is coiled and/or includes one or more bends therein to conserve space and/or accommodate a long capillary tube.

In example embodiments, the capillary tube 18 is formed of or includes a conductive material, and thus acts as its own heater 19 by passing current through the tube. The capillary tube 18 may be any electrically conductive material capable of being resistively heated, while retaining the necessary structural integrity at the operating temperatures experienced by the capillary tube 18, and which is non-reactive with the liquid material. Suitable materials for forming the capillary tube 18 are one or more of stainless steel, copper, copper alloys, porous ceramic materials coated with film resistive material, Inconel® available from Special Metals Corporation, which is a nickel-chromium alloy, nichrome, which is also a nickel-chromium alloy, and combinations thereof.

In one embodiment, the capillary tube 18 is a stainless steel capillary tube 18, which serves as a heater 19 via electrical leads 26 attached thereto for passage of direct or alternating current along a length of the capillary tube 18. Thus, the stainless steel capillary tube 18 is heated by resistance heating. The stainless steel capillary tube 18 may be circular in cross section and may be formed of or include tubing suitable for use as a hypodermic needle of various gauges. For example, the capillary tube 18 may comprise a 32 gauge needle has an internal diameter of 0.11 mm and a 26 gauge needle has an internal diameter of 0.26 mm.

In another embodiment, the capillary tube 18 may be a non-metallic tube such as, for example, a glass tube. In such an embodiment, the heater 19 is formed of or includes a conductive material capable of being resistively heated, such as, for example, stainless steel, nichrome or platinum wire, arranged along the glass tube. When the heater arranged along the glass tube is heated, liquid material in the capillary tube 18 is heated to a temperature sufficient to at least partially volatilize liquid material in the capillary tube 18.

In one embodiment, at least two electrical leads 26 (FIG. 2) are bonded to a metallic capillary tube 18. In an example embodiment, the at least two electrical leads 26 are coupled to the capillary tube 18. In one embodiment, one electrical lead 26 is coupled to a first, upstream portion 101 of the capillary tube 18 and a second electrical lead 26 is coupled to a downstream, end portion 102 of the capillary tube 18, as shown in FIGS. 2 and 3.

In operation, once the capillary tube 18 of FIGS. 2 and 3 is heated, the liquid material contained within a heated portion of the capillary tube 18 is volatilized and ejected out of the outlet 63 where the liquid material expands and mixes with air and forms an aerosol in a mixing chamber 240.

As discussed above and illustrated in FIG. 4, the liquid aerosol formulation can also be used in an electronic smoking article including a heater zone having at least one heater 319 and a filamentary wick 328. The first section 70 includes an outer tube (or casing) 22 extending in a longitudinal direction and an inner tube (or chimney) 362 coaxially positioned within the outer tube 22. In one embodiment, a nose portion 361 of an upstream gasket (or seal) 320 is fitted into an upstream end portion 365 of the inner tube 362, while at the same time, an outer perimeter 367 of the gasket 320 provides a liquid-tight seal with an interior surface 397 of the outer casing 22. The upstream gasket 320 also includes a central, longitudinal air passage 315, which opens into an interior of the inner tube 362 that defines a central channel 321. A transverse channel 333 at an upstream portion of the gasket 320 intersects and communicates with the central, longitudinal air passage 315 of the gasket 320. This channel 333 assures communication between the central, longitudinal air passage 315 and a space 335 defined between the gasket 320 and a threaded connection 74.

In one embodiment, a nose portion 393 of a downstream gasket 310 is fitted into a downstream end portion 381 of the inner tube 362. An outer perimeter 382 of the gasket 310 provides a substantially liquid-tight seal with an interior surface 397 of the outer casing 22. The downstream gasket 310 includes a central channel 384 disposed between the central passage 321 of the inner tube 362 and the mouth end insert 20.

In this example embodiment, the liquid supply reservoir 314 is contained in an annulus between an inner tube 362 and an outer casing 22 and between the upstream gasket 320 and the downstream gasket 310. Thus, the liquid supply reservoir 314 at least partially surrounds the central air passage 321. The liquid supply reservoir 314 comprises a liquid material and optionally a liquid storage medium (not shown) configured to store the liquid material therein.

The inner tube 362 has a central air passage 321 extending therethrough and that houses the heater 319. The heater 319 is in contact with the filamentary wick 328, which preferably extends between opposing sections of the liquid supply reservoir 314 so as to deliver the liquid aerosol formulation from the liquid supply reservoir to the heater 319.

In one embodiment, the electronic smoking article 60 of each embodiment described herein also includes at least one air inlet 440. As shown in FIG. 4, the at least one air inlet 440 can be located upstream of the heater 319.

In the example embodiments illustrated in FIGS. 2 and 3, the at least one air inlet 440 is preferably arranged downstream of the capillary tube 18 so as to minimize drawing air along the capillary tube and thereby avoid cooling of the capillary tube 18 during heating cycles.

In the example embodiments, the at least one air inlet 440 includes one or two air inlets. Alternatively, there may be three, four, five or more air inlets. Altering the size and number of air inlets 440 can also aid in establishing the resistance to draw of the electronic smoking article 60.

The power supply 12 of example embodiments can include a battery or power supply 12 arranged in the electronic smoking article 60. The power supply 12 is configured to apply voltage across the heater 19 associated with the capillary tube 18, as shown in FIGS. 2 and 3, or the heater 319 associated with the wick 328, as shown in FIG. 4. Thus, the heater 19 or 319 volatilizes liquid material according to a power cycle of either a predetermined time period, such as a 2 to 10 second period.

In one embodiment, the electrical contacts or connection between the heater 19/319 and the electrical leads 26 are substantially conductive and temperature resistant while the heater 19/319 is substantially resistive so that heat generation occurs primarily along the heater 19 and not at the contacts.

The battery 12 can be a lithium-ion battery or one of its variants, for example a lithium-ion polymer battery. Alternatively, the battery may be a nickel-metal hydride battery, a nickel cadmium battery, a lithium-manganese battery, a lithium-cobalt battery or a fuel cell. In that case, preferably, the electronic smoking article 60 is usable by a smoker until the energy in the power supply is depleted. Alternatively, the power supply 12 may be rechargeable and include circuitry allowing the battery to be chargeable by an external charging device. In that case, preferably the circuitry, when charged, provides power for a pre-determined number of puffs, after which the circuitry must be re-connected to an external charging device.

In one embodiment, the electronic smoking article 60 of each embodiment also includes control circuitry which can be on a printed circuit board 11 (shown in FIGS. 2, 3 and 4). The control circuitry 11 can also include a heater activation light 27 that is configured to glow when the heater 19/319 is activated. In one embodiment, the heater activation light 27 comprises at least one LED and is at an upstream end 28 (shown in FIG. 1) of the electronic smoking article 60 so that the heater activation light 27 illuminates a cap which takes on the appearance of a burning coal during a puff. Moreover, the heater activation light 27 can be configured to be visible to the smoker. In addition, the heater activation light 27 can be utilized for smoking article system diagnostics. The light 27 can also be configured such that the smoker can activate and/or deactivate the light 27 when desired, such that the light 27 would not activate during smoking if desired.

The time-period of the electric current supply to the heater 19 may be pre-set depending on the amount of liquid desired to be vaporized. The control circuitry 11 can be programmable and can include an application specific integrated circuit (ASIC). In other example embodiments, the control circuitry 11 can include a microprocessor programmed to carry out functions such as heating the capillary tubes and/or operating the valves.

As shown in FIGS. 2, 3 and 4, the electronic smoking article 60 further includes a mouth-end insert 20 having at least two off-axis, preferably diverging outlets 21. In one embodiment, the mouth-end insert 20 includes at least two diverging outlets 21 (e.g., 3, 4, 5, 6 to 8 outlets or more). In one embodiment, the outlets 21 of the mouth-end insert 20 are located at ends of off-axis passages 23 and are angled outwardly in relation to the longitudinal direction of the electronic smoking article 60 (i.e., divergently). As used herein, the term “off-axis” denotes at an angle to the longitudinal direction of the electronic smoking article. Also preferably, the mouth-end insert (or flow guide) 20 includes outlets uniformly distributed around the mouth-end insert 20 so as to substantially uniformly distribute aerosol in a smoker's mouth during use.

In addition, the outlets 21 and off-axis passages 23 are arranged such that droplets of unaerosolized liquid material carried in the aerosol impact interior surfaces of the mouth-end insert 20 and/or interior surfaces of the off-axis passages 23 such that the droplets are removed or broken apart.

In one embodiment, one or more of the outlets 21 may have a diameter of about 0.015 inch to about 0.090 inch (e.g., about 0.020 inch to about 0.040 inch or about 0.028 inch to about 0.038 inch). The size of the outlets 21 and off-axis passages 23 along with the number of outlets 21 can be selected to adjust the resistance to draw (RTD) of the electronic smoking article 60, if desired.

In one embodiment, the electronic smoking article 60 is about the same size as a conventional cigarette. In some embodiments, the electronic smoking article 60 can be about 80 mm to about 110 mm long, preferably about 80 mm to about 100 mm long and about 7 mm to about 10 mm in diameter. For example, in one embodiment, the electronic smoking article is about 84 mm long and has a diameter of about 7.8 mm.

The outer cylindrical housing 22 of the electronic smoking article 60 may be formed of or include any suitable material or combination of materials. In one embodiment, the outer cylindrical housing 22 is formed at least partially of metal and is part of the electrical circuit.

In one embodiment, the liquid aerosol formulation for use in each of the electronic smoking articles 60 described herein includes an aerosol former, optionally water, nicotine, and an acid.

As used herein, the term “aerosol former” describes any suitable known compound or mixture of compounds that, in use, facilitates formation of an aerosol and that is substantially resistant to thermal degradation at the operating temperature of the aerosol-generating article. Suitable aerosol-formers include, but are not limited to, polyhydric alcohols, such as propylene glycol, triethylene glycol, 1,3-butanediol and glycerin; esters of polyhydric alcohols, such as glycerol mono-, di- or triacetate; and aliphatic esters of mono-, di- or polycarboxylic acids, such as dimethyl dodecanedioate and dimethyl tetradecanedioate. Preferred aerosol formers are polyhydric alcohols or mixtures thereof, such as propylene glycol, triethylene glycol, 1,3-butanediol and glycerin. The aerosol-forming substrate may comprise a single aerosol former. Alternatively, the aerosol-forming substrate may comprise a combination of two or more aerosol formers.

In one embodiment, the aerosol former is one of propylene glycol, glycerin and combinations thereof. In another embodiment, the aerosol former is glycerin. In one embodiment, the aerosol former is included in an amount ranging from about 40% by weight based on the weight of the liquid formulation to about 90% by weight based on the weight of the liquid formulation (e.g., about 50% to about 80%, about 55% to about 75% or about 60% to about 70%). Moreover, in one embodiment, the liquid formulation can include propylene glycol and glycerin included in a ratio of about 3:2. In one embodiment, the ratio of propylene glycol and glycerin may be substantially 2:3 and 3:7.

The liquid formulation optionally includes water. Water can be included in an amount ranging from about 5% by weight based on the weight of the liquid formulation to about 40% by weight based on the weight of the liquid formulation, more preferably in an amount ranging from about 10% by weight based on the weight of the liquid formulation to about 15% by weight based on the weight of the liquid formulation.

The liquid aerosol formulation optionally includes a flavorant in an amount ranging from about 0.01% to about 15% by weight (e.g., about 1% to about 12%, about 2% to about 10%, or about 5% to about 8%). The flavorant can be a natural flavorant or an artificial flavorant. In one embodiment, the flavorant is one of tobacco flavor, menthol, wintergreen, peppermint, herb flavors, fruit flavors, nut flavors, liquor flavors, and combinations thereof.

In one embodiment, the liquid aerosol formulation includes one of more acids from pyruvic acid, formic acid, oxalic acid, acetic acid, isovaleric acid, valeric acid, propionic acid, octanoic acid, lactic acid, levulinic acid, sorbic acid, malic acid, tartaric acid, succinic acid, citric acid, benzoic acid, oleic acid, aconitic acid, butyric acid, cinnamic acid, decanoic acid, 3,7-diemthyl-6-octenoic acid, 1-glutamic acid, heptanoic acid, hexanoic acid, 3-hexenoic acid, trans-2-hexenoic acid, isobutyric acid, lauric acid, 2-methylbutyric acid, 2-methylvaleric acid, myristic acid, nonanoic acid, palmitic acid, 4-pentenoic acid, phenylacetic acid, 3-phenylpropionic acid, hydrochloric acid, phosphoric acid, sulfuric acid and combinations thereof. The acid also may be incorporated into the liquid aerosol formulation in the form of a salt. In one embodiment, the salt form of the acid is selected such that the addition of the acid does not have significant adverse effects on aerosol transfer efficiency and/or the reaction of the corresponding free acid form with nicotine.

The acids included in the liquid aerosol formulation can have a boiling point of at least about 100° C. For example, the acids can have a boiling point ranging from about 100° C. to about 300° C., more preferably about 150° C. to about 250° C. (e.g., about 160° C. to about 240° C., about 170° C. to about 230° C., about 180° C. to about 220° C. or about 190° C. to about 210° C.). By including acids having a boiling point within this range, the acid may volatilize when heated by heater elements of electronic smoking articles as previously described. In one embodiment utilizing a heater coil and a wick, the heater coil may reach an operating temperature at or about 300° C.

In one embodiment, the acid is included in the liquid aerosol formulation in an amount sufficient to reduce the pH of the liquid aerosol formulation in the range of about 3 to about 8. In some embodiments, the acid is included in the liquid aerosol formulation in an amount sufficient to adjust the pH of the liquid aerosol formulation in the range of about 3 to about 5. In some other embodiments, the acid is included in the liquid aerosol formulation in an amount sufficient to adjust the pH of the liquid aerosol formulation in the range of about 7 to about 8. Moreover, the acid may be condensable at ambient temperature (except for HCl and other acids which are gases at ambient temperature).

The total acid content of the liquid aerosol formulation preferably ranges from about 0.1% by weight to about 6% by weight, and more preferably from about 0.1% by weight to about 2% by weight, based on the weight of the liquid aerosol formulation containing about 3% by weight nicotine. The liquid aerosol combination may also contain between about 4.5% and 5% nicotine by weight. In one embodiment, the total acid content of the liquid aerosol formulation is less than about 3% by weight based on the weight of the liquid aerosol formulation containing about 3% by weight nicotine. In another embodiment, the total acid content of the liquid aerosol formulation is less than about 0.5% by weight based on the weight of the liquid aerosol formulation containing about 3% by weight nicotine. The liquid aerosol combination may also contain between about 4.5% and 5% nicotine by weight. When hydrochloric acid, phosphoric acid, and/or sulfuric acid is used, the total acid content of the liquid aerosol formulation is preferably about 0.05% by weight to about 2% by weight, and more preferably about 0.1% by weight to about 1% by weight, based on the weight of the liquid aerosol formulation containing about 3% by weight nicotine.

In one embodiment, the acid comprises at least one of pyruvic acid, lactic acid, benzoic acid and acetic acid. In a further embodiment, the combination of pyruvic acid, lactic acid, benzoic acid and optionally acetic acid is included in a liquid formulation of an electronic smoking article, which combination produced the beneficial effects previously associated with use of nicotine bitartrate (or the addition of tartaric acid) in a liquid formulation for an electrical smoking article, but without the production of char and/or a polymeric residue associated with use of nicotine bitartrate (or tartaric acid).

In one embodiment, pyruvic acid is included in an amount ranging from about 0.1% by weight to about 5.0% by weight based on the weight of the liquid aerosol formulation containing about 3% by weight nicotine. Lactic acid can be included in the liquid aerosol formulation in an amount ranging from about 0.1% by weight to about 5.0% by weight based on the weight of the liquid aerosol formulation containing about 3% by weight nicotine. Benzoic acid can be included in an amount ranging from about 0.1% by weight to about 5.0% by weight based on the weight of the liquid aerosol formulation containing about 3% by weight nicotine. Acetic acid can be included in an amount ranging from about 0.1% by weight to about 5.0% by weight based on the weight of the liquid aerosol formulation containing about 3% by weight nicotine. In one embodiment, pyruvic acid is included in an amount of about 0.5% by weight based on the weight of the liquid aerosol formulation containing about 3% by weight nicotine, lactic acid is included in an amount of about 0.4% by weight based on the weight of the liquid aerosol formulation containing about 3% by weight nicotine, and benzoic acid is included in an amount of about 0.1% by weight based on the weight of the liquid aerosol formulation containing about 3% by weight nicotine. In one embodiment, the entire acid content of the liquid aerosol formulation is less than about 3% by weight based on the weight of the liquid aerosol formulation containing about 3% by weight nicotine.

In another embodiment, the acid comprises at least one of pyruvic acid, formic acid, oxalic acid, and glycolic acid. In a further embodiment, the combination of pyruvic acid, formic acid, oxalic acid, and glycolic acid is included in a liquid formulation of an electronic smoking article.

In one embodiment, pyruvic acid can be included in the liquid aerosol formulation in an amount ranging from about 0.0001% by weight to about 2.0% by weight based on the weight of the liquid aerosol formulation containing about 3% by weight nicotine. Formic acid is included in an amount ranging from about 0.0001% by weight to about 2.0% by weight % by weight based on the weight of the liquid aerosol formulation containing about 3% by weight nicotine. Oxalic acid and glycolic acid can be included in an amount ranging from about 0.0001% by weight to about 2.0% by weight based on the weight of the liquid aerosol formulation containing about 3% by weight nicotine.

In one embodiment, pyruvic acid is included in an amount of about 0.35% by weight based on the weight of the liquid aerosol formulation containing about 3% by weight nicotine, formic acid is included in an amount of about 0.036% by weight based on the weight of the liquid aerosol formulation containing about 3% by weight nicotine, oxalic acid is included in an amount of about 0.013% by weight based on the weight of the liquid aerosol formulation containing about 3% by weight nicotine, and glycolic acid is included in an amount of about 0.05% by weight based on the weight of the liquid aerosol formulation containing about 3% by weight nicotine. With inclusion of these acids at these levels, a liquid aerosol formulation of 3% by weight nicotine (molecular form) produced an aerosol which exhibited acceptable low to moderate throat harshness. The liquid aerosol formulation can include pyruvic acid, formic acid, oxalic acid and glycolic acid in a ratio of 9.72:1.00: 0.36:1.36. In one embodiment, the entire acid content of the liquid aerosol formulation is less than about 0.5% by weight based on the weight of the liquid aerosol formulation containing about 3% by weight nicotine.

The following experiments revealed the differences among a Control formulation (3 wt % nicotine with no acid), a nicotine bitartrate-containing liquid aerosol formulation (3 wt % nicotine achieved with addition of 8.6 wt % nicotine bitartrate (nicotine bitartrate being about 35 wt % nicotine and about 65 wt % tartaric acid)) (“Sample A”), and a liquid aerosol formulation containing 3 wt % nicotine, 0.036 wt % formic acid, 0.35 wt % pyruvic acid, 0.013 wt % oxalic acid, and 0.05 wt % glycolic acid (“Sample B” having an “Acid Complex No. 1”). In Sample B, the total amount of acids is 0.45 wt %. The carrier in Control, Sample A and Sample B is a mixture of 15 wt % water, and 85 wt % of propylene glycol and glycerin in a weight ratio of 40:60.

The Control formulation, Sample A and Sample B were tested by using a smoking machine having E-vapor smoking protocol. The E-vapor smoking protocol includes:
puff volume (mL): 55.0±0.3
puff duration: (sec): 5.0±0.02
puff frequency (sec): 30
puff profile: square wave profile

The puff volume is the volume of air that is drawn through the e-cigarette during the puffing action. The duration is the length of the puff, in seconds, and the frequency is the amount of time between successive puffs, also in seconds. Square wave profile refers to the shape of the puff profile's volumetric flow rate vs. time plot (this profile is a preset profile that is controlled by the smoking machine). Samples are usually smoked to exhaustion (meaning battery failure), which is to about 80 puffs.

Samples to be smoked were mounted into a holder that positions a filter directly behind the mouthpiece. The majority of the aerosol formed during each puff (all of the particulate phase) is trapped on this filter pad. “20 puff collections” means that 20 puffs are collected on a single pad. After each 20 puff collection, the pad is weighed to determine aerosol mass and total nicotine delivery by extraction and analysis (e.g., by GC-MS). At that point, a new pad is installed, and the next group of 20 puffs is collected, until battery exhaustion (again, about 80 puffs).

Residue is determined gravimetrically. First, the smoking devices are disassembled so that the wick/coil element can be removed. Once removed, the wick/coil is swirled gently in methanol to remove excess liquid, and then air dried. Next, the ends of the wick are trimmed to form a small section of wick within the coil that can easily fit within a TGA (thermogravimetric analyzer) pan. The TGA method heats the samples under nitrogen to 250° C., and then maintains that temperature for 3 minutes. During this temperature ramp and 3 minute isothermal period, weight change is associated with the evolution of volatile species from the wick. At the end of the three minute isothermal period, the sample mass has stabilized. Next, the atmosphere in the TGA is switched to air to allow for oxidation of non-volatile species. The TGA further heats the sample to 800° C., then holds isothermally for another 3 minutes. During this time, there is weight loss that is associated with oxidation of non-volatile species. At the end of this three minute isothermal segment, again the sample mass has stabilized. At the end of the experiment, the weight loss that occurs between the two isothermal segments is determined, and this weight loss is reported as the residue.

To determine the amount of gas phase nicotine, it is necessary to separate the particulate phase and gas phase of the aerosol that is produced by actuation of the e-cigarette device. For this purpose, a polytetrafluoroethylene (PTFE) filter with a 2 μm pore size is used. Gas phase nicotine is detected by mass spectroscopy. The mass spectrometer is operated in EI (electron ionization) mode, and the detector is operated in SIM mode (selective ion mode). The mass fragments monitored are 84 atomic mass unit (amu) (dominant peak in nicotine mass spectrum), 162 amu (parent peak in nicotine pass spectrum), and 133 amu (secondary peak in nicotine mass spectrum). Additionally, the 61 amu peak (from glycerine mass spectrum) is monitored to ensure that there is no breakthrough of the particulate phase of the aerosol through the PTFE filter pad.

The e-cigarette sample is mounted in a sample holder with the PTFE filter positioned behind the mouthpiece of the e-cigarette device. Smoking is performed by an automated smoking machine, with the following puff parameters: 55 cc puff volume, 4 second puff duration, square wave puff profile, 30 second interpuff interval. Upon actuation of the e-cigarette device by the smoking machine, the particulate phase is trapped on the PTFE pad, while the gas phase is allowed to pass through the pad to the automated sampling loop (operated at 200° C.). The automated sampling loop is switched from sampling mode to injection mode, at which point a 2 cc volume of the gas phase is injected through a guard column (operated at 250° C.) to the mass spectrometer for detection of gas phase nicotine. Generally 10 puffs per replicate are analyzed.

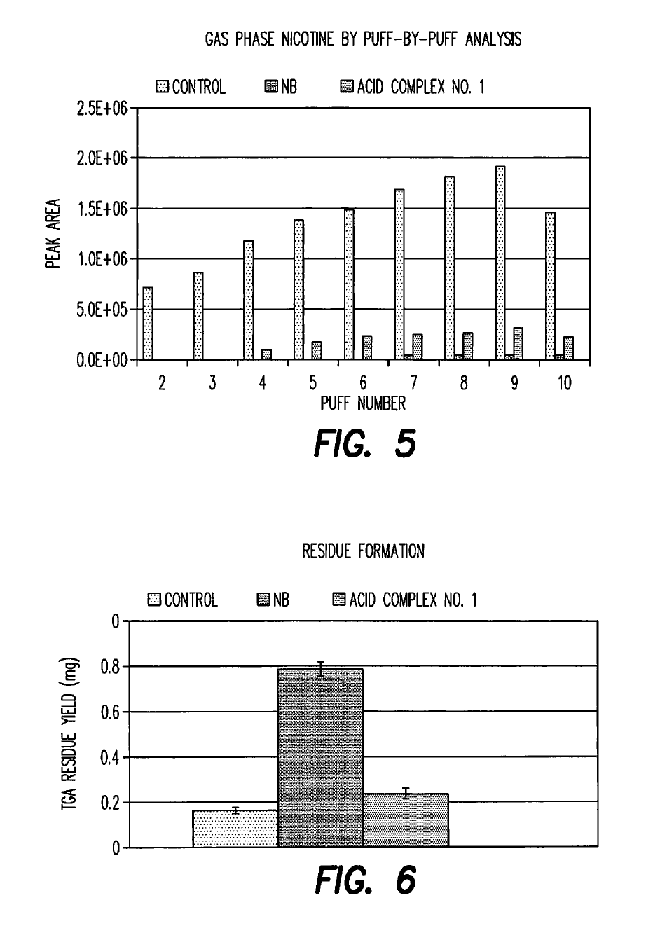

FIG. 5 shows gas phase nicotine contents by puff-by-puff analysis. The results show that both Sample A and Sample B generated significantly reduced amounts of gas phase nicotine compared to the Control formulation. Further, Sample A resulted in more reduction than Sample B. Throughout the puff numbers, the level of gas phase nicotine produced with Sample B was not greater than about 15% of the gas phase nicotine produced with the Control formulation.

FIG. 6 shows residue formation of the Control formulation, Sample A and Sample B. The results show that Sample B generated the residue formation in an amount comparable to the Control formulation. However, Sample A generated a significantly more residue formation than Sample B and the Control formulation.

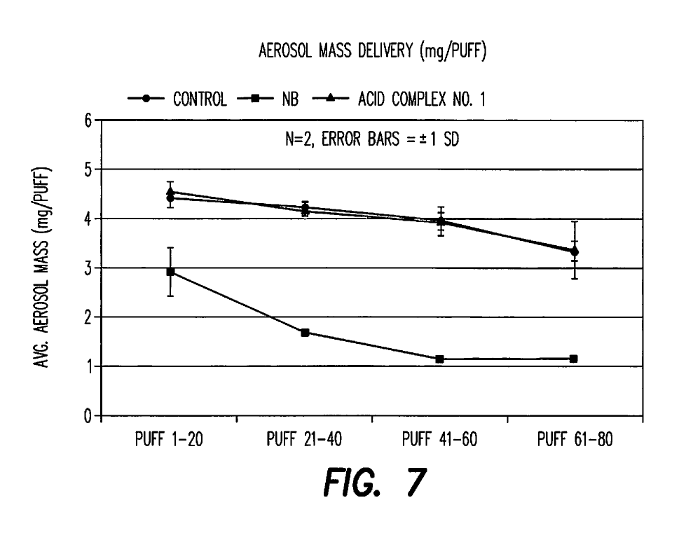

FIG. 7 shows particulate phase aerosol mass delivery of the Control formulation, Sample A and Sample B. The results show that the Control formulation and Sample B had similar aerosol mass delivery per puff. Aerosol mass delivery with Sample B decreases significantly during the first 20 puffs and beyond. It is understood that the total aerosol (gas phase and particulate phase) mass delivery has the similar trend as the aerosol (particulate phase) mass delivery.

Other combinations of acids may be constructed by finding substitutes for each or a subset of the above listed acids. In other words, some or all of the acids in the above combination may be replaced or supplemented with acids having similar physical/chemical properties, such as boiling point, polarity, pKa, volatility (such that they volatilize when heated in an electronic smoking article) and sensory properties (such as a lack of sensory off-notes at their level of inclusion).

For example, additional combinations of acids may be suitable by using substitutes in a similar manner for the latter three acids in the combination of pyruvic acid, formic acid, oxalic acid and glycolic acid.

In one embodiment, if some of the acids in an acid combination have a sensorial or other property that other acids lack, proportional inclusion rates of those acids with the lacking properties may be minimized.

The acid combination included in the liquid aerosol formulation preferably reduces gas phase nicotine when the liquid aerosol formulation is heated by the heater of an electronic smoking article.

In some embodiments, the amount of acid to be added to the liquid aerosol formulation may depend on the strength of the acid and the amount needed to adjust the pH of the liquid aerosol formulation to the desired range. If too much acid is added, essentially all of the available nicotine will be protonated and will enter the particulate phase of the aerosol, leaving very little unprotonated nicotine in the gas phase of the aerosol. The resultant aerosol may not produce sufficient levels of sensory response in terms of low to moderate throat harshness to meet preferences of the smoker of lit-end cigarettes. In contrast, if too little acid or an ineffective (weak) acid is added, a larger amount of nicotine will remain unprotonated and in the gas phase of the aerosol, such that the smoker will experience increased and possibly objectionable throat harshness. With liquid formulations having a nicotine content above approximately 2% by weight, and in the absence of addition of the combinations of acids described herein, perceived throat harshness may approach levels which render the aerosol unpleasant when inhaled. With liquid formulations of nicotine content above approximately 4% by weight, and in the absence of the combination of acids as described herein, perceived throat harshness may approach levels rendering the aerosol uninhalable. With the addition of acids according to the teachings herein, perceived throat harshness is maintained at desirable levels, akin to that experienced with lit-end cigarettes.

In one embodiment, the nicotine is added to the liquid aerosol formulation in the molecular (unprotonated, uncharged) form of nicotine, which is known to be basic (in the acid-base context). Alternatively, the nicotine is added to the formulation as a salt. The nicotine may be added before or after the addition of an acid.

In one embodiment, the nicotine is added before the addition of an acid. The nicotine is included in the liquid aerosol formulation in an amount ranging from about 1% by weight to about 10% by weight (e.g., about 2% to about 9%, about 2% to about 8%, about 2% to about 6%) based on the total weight of the liquid aerosol formulation. In one embodiment, the nicotine is added in an amount of up to about 5% by weight based on the total weight of the liquid aerosol formulation. In one embodiment, the nicotine content of the liquid aerosol formulation is about 2% by weight or greater based on the total weight of the liquid aerosol formulation. In another embodiment, the nicotine content of the liquid aerosol formulation is about 3% by weight or greater based on the total weight of the liquid aerosol formulation.

Typically, molecular nicotine in an aqueous solution has a pH of about 9 to about 10. Thus, the acids would preferably be added in an amount sufficient to reduce the pH in the range of about 3 to about 8. In one embodiment, molecular nicotine is added in liquid form.

When vaporized in the electronic smoking article, the liquid aerosol formulation is capable of forming an aerosol having a particulate phase and a gas phase. In one embodiment, the particulate phase contains protonated nicotine and the gas phase contains unprotonated nicotine. In another example embodiment, the majority of nicotine is protonated and in the particulate phase, while a minority amount of nicotine is contained in the gas phase. Once the liquid aerosol formulation has been vaporized, the vapor condenses, nicotine is protonated and particles including the protonated nicotine are formed. A minor amount of the nicotine remains unprotonated and stays in the gas phase of the newly generated aerosol. In one embodiment, the acid is included in an amount sufficient to reduce the amount of nicotine gas phase component to a level not greater than about 30%, preferably not greater than about 20%, and more preferably not greater than about 15%, of the level of nicotine gas phase component produced without the acid. In one embodiment, the particulate phase includes particles ranging in size from about 0.2 micron to about 2 microns.

The addition of the acids as taught herein may allow the acids to enter the initial, not fully developed aerosol-vapor system, when the liquid is vaporized by the heater of an electronic cigarette. The acids survive the heating, and remain available to protonate nicotine so that most, if not almost all, of the nicotine remains and/or enters the particulate phase as the aerosol develops, because the nicotine is charged (protonated). As with an aerosol produced by a lit end cigarette, the initial gas phase nicotine content of the electronically produced aerosol is quite low, preferably the amount of nicotine gas phase component is reduced by about 30% by weight or greater, preferably about 60% by weight to about 70% by weight, more preferably about 70% by weight or greater, and most preferably 85% by weight or greater, of the level of nicotine gas phase component produced without the acid. Additionally, the nicotine residing in the particulate phase is predominantly protonated and therefore charged and mostly unavailable for transfer into the gas phase of the aerosol.

Furthermore, the acids may be selected and the concentrations thereof may be adjusted to maintain the aforementioned, desired low levels of gas phase nicotine, even at the more elevated nicotine content levels in the liquid formulation.

By providing a liquid formulation comprising a nicotine at levels of greater than 2% or more by weight, more preferably in range of 2% to about 6% by weight, together with an addition of the acids to the liquid formulation in accordance with the teachings herein, the perceived sensory benefits associated with the higher nicotine levels is achieved (warmth in the chest), while also avoiding the sensory deficits previously associated with higher nicotine levels (excessive harshness in the throat), thereby providing adult, lit-end cigarette smokers an electronic cigarette that provides a sensorially pleasant smoking experience, including a low to moderate throat harshness response in the throat and a perceived warmth in the chest.

With the improved liquid formulation, much of the nicotine in the particulate phase is protonated by the presence of the acids, and any nicotine that is removed from the gas phase by absorption in the throat is not readily replaced by nicotine from the particulate phase. Instead, the protonated nicotine remains in the particulate phase and is not allowed to elevate the harshness response to unacceptable levels. An aerosol produced under the teachings herein can provide enjoyable sensations from low to mild throat harshness, generally within the expectations of smokers of lit end cigarettes, even with liquid formulations of elevated nicotine content.

In terms of smoking enjoyment, enjoyable sensations are experienced at low to mild levels of throat harshness.

Advantageously, the addition of the acids described herein reduces throat harshness during both inhalation, while substantially abating charring of the heater and the wick.

The teachings herein may be applicable to all or a substantial number of forms of electronic smoking articles, such as electronic cigarettes, e-vaping devices, cigars, pipes, hookas and others, regardless of their size and shape.

In example embodiments, the total acid content of the liquid aerosol formulation ranges from about 0.1% by weight to about 6% by weight, such as from about 0.5% to about 4% by weight, or from about 1% to about 3% by weight, or from about 1.5% to about 2.5% by weight, or from about 0.1% by weight to about 2% by weight. For example, in embodiments, the total acid content of the liquid aerosol formulation may be from about 0.5% to about 2.5%, such as from about 1.5% to about 2.0% by weight based on the total weight of the liquid aerosol formulation, where the liquid aerosol formulation may contain from about 2 to about 5% nicotine, such as from about 2.5% to about 4.5% nicotine.

In embodiments, pyruvic acid is included in an amount ranging from about 0.1 to about 2% by weight based on the weight of the liquid aerosol formulation; lactic acid is included in an amount ranging from about 0.1 to about 2% by weight based on the weight of the liquid aerosol formulation; benzoic acid is included in an amount ranging from about 0.1 to about 2% by weight based on the weight of the liquid aerosol formulation; and acetic acid is included in an amount ranging from about 0.1 to about 2% by weight based on the weight of the liquid aerosol formulation. In embodiments, a combination of pyruvic acid, lactic acid, benzoic acid and acetic acid is present in the liquid aerosol formulation in a total amount from about 0.1 to about 2% by weight based on the weight of the liquid aerosol formulation, such as from about 1.5 to about 2% by weight. In embodiments, pyruvic acid, lactic acid, benzoic acid, and acetic acid are each added, preferably in approximately equal amounts (equal by weight percent of the liquid formulation). The formulation may contain nicotine in an amount ranging from about 2% by weight to about 10% by weight, such as from about 2% to about 9%, or from about 2% to about 8%, or from about 2% to about 6%, or from about 2% to about 5%. For example, in embodiments, the formulation may contain nicotine in an amount from about 2.5% to about 4.5% based on the total weight of the liquid aerosol formulation.

In embodiments, the nicotine is included in the liquid aerosol formulation in an amount ranging from about 2% by weight to about 10% by weight (e.g., about 2% to about 9%, about 2% to about 8%, about 2% to about 6%, about 2% to about 5%, about 2.5% to about 4.5%) based on the total weight of the liquid aerosol formulation. In one embodiment, the nicotine is added in an amount of up to about 5% by weight based on the total weight of the liquid aerosol formulation. In one embodiment, the nicotine content of the liquid aerosol formulation is about 2% by weight or greater based on the total weight of the liquid aerosol formulation. In another embodiment, the nicotine content of the liquid aerosol formulation is about 2.5% by weight or greater based on the total weight of the liquid aerosol formulation. In another embodiment, the nicotine content of the liquid aerosol formulation is about 3% by weight or greater based on the total weight of the liquid aerosol formulation. In another embodiment, the nicotine content of the liquid aerosol formulation is about 4% by weight or greater based on the total weight of the liquid aerosol formulation. In another embodiment, the nicotine content of the liquid aerosol formulation is about 4.5% by weight or greater based on the total weight of the liquid aerosol formulation. The following examples of electronic cigarettes are discussed:

### EXAMPLE 1

A control electronic cigarette including a rechargeable battery, a light-emitting diode (LED) light, a heating coil, and a cartomizer was prepared. The cartomizer contained 420 mg propylene glycol/glycerin/water based solution (wherein the solution contained 15% water and a 30/70 proportion of propylene glycol/glycerin), 1% by weight flavorant, 2.5% nicotine by weight (NBW) tobacco-derived nicotine, and 0% acid. For example, for menthol applications, the flavorant may be included at one or more concentrations up to substantially 4% by weight.

### EXAMPLE 2

An electronic cigarette including a rechargeable battery, a light-emitting diode (LED) light, a heating coil, and a cartomizer was prepared. The cartomizer contained 420 mg propylene glycol/glycerin (30/70) based solution, 15% water, 1% by weight flavorant, 3.0% (NBW) tobacco-derived nicotine and 1.5% by weight of a combination of benzoic acid, lactic acid, acetic acid, and pyruvic acid in equal proportions (each acid being 0.375% by weight of the total weight of the aerosol formulation). For example, for menthol applications, the flavorant may be included at one or more concentrations up to substantially 4% by weight.

### EXAMPLE 3

An electronic cigarette including a rechargeable battery, a light-emitting diode (LED) light, a heating coil, and a cartomizer was prepared. The cartomizer contained 420 mg propylene glycol/glycerin (30/70) based solution, 15% water, 1% by weight flavorant, 4.5% (NBW) tobacco-derived nicotine, and 1.5% by weight of a combination of benzoic acid, lactic acid, acetic acid, and pyruvic acid in equal proportions (each acid being 0.375% by weight of the total weight of the aerosol formulation). For example, for menthol applications, the flavorant may be included at one or more concentrations up to substantially 4% by weight.

### EXAMPLE 4

An electronic cigarette including a rechargeable battery, an LED light, a heating coil, and a cartomizer was prepared. The cartomizer contained 420 mg propylene glycol/glycerin (30/70) based solution, 15% water, 1% by weight flavorant, 4.5% (NBW) tobacco-derived nicotine, and 2.0% by weight of a combination of benzoic acid, lactic acid, acetic acid, and pyruvic acid in equal proportions (each acid being 0.5% by weight percent of the liquid formulation). For example, for menthol applications, the flavorant may be included at one or more concentrations up to substantially 4% by weight.

Comparing Example 1 to Examples 2-4, the acid addition in Examples 2-4 is expected to reduce an amount of perceived throat harshness in comparison to the aerosolized formulation being formed in Example 1.

Additional examples include liquid aerosol formulations wherein:

### EXAMPLE 5

the concentration of the nicotine is between substantially 1.5% by weight and substantially 6% by weight, between substantially 3% by weight and substantially 4.5% by weight, or between substantially 3% by weight and substantially 5% by weight;

### EXAMPLE 6

the concentration of the acidic compound is between substantially 0.25% by weight and substantially 2% by weight, between substantially 0.5% by weight and substantially 1.5% by weight, or between substantially 1.5% by weight and substantially 2% by weight;

### EXAMPLE 7

the acidic compound may include between 2 and 10 acids, and may include 4 acids;

### EXAMPLE 8

the acidic compound may also include substantially equal parts of each individual acid included in the compound such as pyruvic acid, lactic acid, benzoic acid and acetic acid;

### EXAMPLE 9

the liquid aerosol formulation includes substantially 3% nicotine by weight, substantially 0.5% pyruvic acid, substantially 0.4% lactic acid and 0.1% benzoic acid;

### EXAMPLE 10

the liquid aerosol formulation includes substantially 3% nicotine by weight, substantially 0.5% pyruvic acid, substantially 0.4% lactic acid and substantially 0.1% hydrochloric acid;

### EXAMPLE 11

the liquid aerosol formulation includes substantially 3% nicotine by weight, substantially 0.5% nicotine bitartrate and substantially 0.2% hydrochloric acid;

### EXAMPLE 12

the liquid aerosol formulation includes substantially 3% nicotine by weight, substantially 0.5% pyruvic acid, substantially 0.4% lactic acid and substantially 0.5% tartaric acid;

### EXAMPLE 13

the liquid aerosol formulation includes substantially 3% nicotine by weight, substantially 0.5% pyruvic acid, substantially 0.4% lactic acid, substantially 0.1% benzoic acid and substantially 0.1% oleic acid;

### EXAMPLE 14

the liquid aerosol formulation includes substantially 3% nicotine by weight, substantially 0.5% nicotine bitartrate, substantially 0.5% pyruvic acid, substantially 0.4% lactic acid and substantially 0.1% benzoic acid;

### EXAMPLE 15

the liquid aerosol formulation includes substantially 3% nicotine by weight, substantially 0.5% nicotine bitartrate, substantially 0.5% pyruvic acid, substantially 0.4% lactic acid, substantially 0.1% benzoic acid and substantially 1% oleic acid;

### EXAMPLE 16

the liquid aerosol formulation includes substantially 3% to 5% nicotine by weight, substantially 0.036% to 0.144% formic acid, substantially 0.35% to 1.4% pyruvic acid, substantially 0.013% to 0.052% oxalic acid and substantially 0.05% to 0.2% glycolic acid;

### EXAMPLE 17

the liquid aerosol formulation includes substantially 3% nicotine by weight, substantially 0.036% formic acid, substantially 0.35% pyruvic acid, substantially 0.013% oxalic acid and substantially 0.05% glycolic acid;

### EXAMPLE 18

the liquid aerosol formulation includes substantially 3% to 5% nicotine by weight, substantially 1% to 2% nicotine bitartrate and substantially 0.4% to 0.9% acetic acid;

### EXAMPLE 19

the liquid aerosol formulation includes substantially 3% nicotine by weight, substantially 1% nicotine bitartrate and substantially 0.4% acetic acid;

### EXAMPLE 20

the liquid aerosol formulation includes substantially 3% to 5% nicotine by weight, substantially 0.5% to 1% nicotine bitartrate and substantially 0.5% to 1% of a mixture of substantially equal parts pyruvic acid, benzoic acid, lactic acid and acetic acid;

### EXAMPLE 21

the liquid aerosol formulation includes substantially 3% to 5% nicotine by weight, substantially 0.5% to 1% nicotine bitartrate and substantially 0.01% to 0.1% hydrochloric acid;

### EXAMPLE 22

the liquid aerosol formulation includes substantially 3% to 5% nicotine by weight, substantially 0.5% to 1% oleic acid and substantially 0.5% to 1.5% of a mixture of substantially equal parts pyruvic acid, benzoic acid, lactic acid and acetic acid;

### EXAMPLE 23

the liquid aerosol formulation includes substantially 3% to 5% nicotine by weight, substantially 0.1% to 0.5% sorbic acid, substantially 0.1% to 0.5% tartaric acid and substantially 0.1% to 0.75% pyruvic acid;

### EXAMPLE 24

when the concentration of nicotine by weight is substantially 5%, hydrochloric acid may be added at a concentration of substantially 0.01% to 0.1%;

### EXAMPLE 25

the liquid aerosol formulation includes substantially 3% to 5% nicotine by weight, substantially 0.1% to 0.5% succinic acid, substantially 0.1% to 0.75% pyruvic acid and substantially 0.1% to 0.5% lactic acid;

### EXAMPLE 26

when the concentration of nicotine by weight is substantially 5%, hydrochloric acid may be added at a concentration of substantially 0.01% to 0.1%, or tartaric acid may be added at a concentration of substantially 0.1% to 0.25%; and/or

### EXAMPLE 27

the acidic compound may also include more pyruvic acid than one or more of the lactic acid, the benzoic acid and the acetic acid.

### EXAMPLE 28

An electronic cigarette including a rechargeable battery, a light-emitting diode (LED) light, a heating coil, and a cartomizer. The cartomizer may include a solution of a combination of propylene glycol and glycerin, water, optionally flavorant, tobacco-derived nicotine and a combination of benzoic acid, lactic acid, acetic acid, and pyruvic acid. For example, the benzoic acid, the lactic acid, the acetic acid and the pyruvic acid are in equal proportions. In addition, the resulting aerosol may include an amount of nicotine in the gas phase of the aerosol of less than or equal to substantially 1% of the total nicotine delivered. The above combination of the benzoic acid, the lactic acid, the acetic acid and the pyruvic acid, together with the nicotine concentration in the gas phase of equal to or less than substantially 1% of the total nicotine delivered, results in an inhalable vapor that has an improved combination of harshness and warmth in chest compared to other combinations of acids and higher concentrations of nicotine in the gas phase.

### EXAMPLE 29

An electronic cigarette including a rechargeable battery, a light-emitting diode (LED) light, a heating coil, and a cartomizer. The cartomizer may include a solution of a combination of propylene glycol and glycerin, water, optionally flavorant, tobacco-derived nicotine and a combination of benzoic acid, lactic acid, acetic acid, and pyruvic acid. For example, for menthol formulations, the concentration of nicotine may be substantially 4.5% by weight, and the concentration of the combination of benzoic acid, lactic acid, acetic acid and pyruvic acid may be between substantially 0.25% and 1% by weight. For example, the concentration of the combination of benzoic acid, lactic acid, acetic acid and pyruvic acid may be substantially 0.5%. In addition, the resulting aerosol may include an amount of nicotine in the gas phase of the aerosol of less than or equal to substantially 1% of the total nicotine delivered. The above combination of the benzoic acid, the lactic acid, the acetic acid and the pyruvic acid, together with the nicotine concentration in the gas phase of equal to or less than substantially 1%, results in an inhalable vapor that has an improved combination of harshness and warmth in chest compared to other combinations of acids and higher concentrations of nicotine in the gas phase.

When the word “about” is used in this specification in connection with a numerical value, it is intended that the associated numerical value include a tolerance of ±10% around the stated numerical value. Moreover, when reference is made to percentages in this specification, it is intended that those percentages are based on weight, i.e., weight percentages. The expression “up to” includes amounts of zero to the expressed upper limit and all values therebetween. When ranges are specified, the range includes all values therebetween such as increments of 0.1%.

Moreover, when the words “generally” and “substantially” are used in connection with geometric shapes, it is intended that precision of the geometric shape is not required but that latitude for the shape is within the scope of the disclosure. Although the tubular elements of the embodiments are preferably cylindrical, other tubular cross-sectional forms are contemplated, such as square, rectangular, oval, triangular and others.

Example embodiments having thus been described, it will be obvious that the same may be varied in many ways. Such variations are not to be regarded as a departure from the intended spirit and scope of example embodiments, and all such modifications as would be obvious to one skilled in the art are intended to be included within the scope of the following claims.

1-70. (canceled)

71. A liquid aerosol formulation of an electronic smoking article, the liquid aerosol formulation comprising:
an aerosol former;
nicotine; and
an acidic compound;
wherein the liquid aerosol formulation is configured to form an aerosol having a particulate phase and a gas phase when heated in the electronic smoking article; and
wherein a concentration of nicotine in the gas phase is equal to or smaller than substantially 1% by weight.

72. The liquid aerosol formulation of claim 71, wherein the acidic compound comprises at least one of pyruvic acid, formic acid, oxalic acid, glycolic acid, acetic acid, isovaleric acid, valeric acid, propionic acid, octanoic acid, lactic acid, sorbic acid, malic acid, tartaric acid, succinic acid, citric acid, benzoic acid, oleic acid, aconitic acid, butyric acid, cinnamic acid, decanoic acid, 3,7-dimethyl-6-octenoic acid, 1-glutamic acid, heptanoic acid, hexanoic acid, 3-hexenoic acid, trans-2-hexenoic acid, isobutyric acid, lauric acid, 2-methylbutyric acid, 2-methylvaleric acid, myristic acid, nonanoic acid, palmitic acid, 4-pentenoic acid, phenylacetic acid, 3-phenylpropionic acid, hydrochloric acid, phosphoric acid and sulfuric acid.

73. The liquid aerosol formulation of claim 71, wherein the acidic compound comprises pyruvic acid, lactic acid, benzoic acid and acetic acid.

74. The liquid aerosol formulation of claim 71, wherein a concentration of the nicotine is between substantially 1.5% by weight and substantially 6% by weight.

75. The liquid aerosol formulation of claim 74, wherein the concentration of the nicotine is between substantially 3% by weight and substantially 4.5% by weight.

76. The liquid aerosol formulation of claim 71, wherein a concentration of the acidic compound is between substantially 0.25% and substantially 2%.

77. The liquid aerosol formulation of claim 76, wherein the concentration of the acidic compound is between substantially 0.5% and substantially 1.5%.

78. The liquid aerosol formulation of claim 76, wherein the concentration of the acidic compound is between substantially 1.5% and substantially 2%.

79. The liquid aerosol formulation of claim 72, wherein the acidic compound comprises between 2 and 10 acids.

80. The liquid aerosol formulation of claim 79, wherein the acidic compound comprises 4 acids.

81. The liquid aerosol formulation of claim 71, wherein the acidic compound comprises at least one of pyruvic acid, acetic acid, isovaleric acid, valeric acid, propionic acid, octanoic acid, lactic acid, sorbic acid, malic acid, succinic acid, citric acid, benzoic acid, oleic acid, aconitic acid, butyric acid, cinnamic acid, decanoic acid, 3,7-dimethyl-6-octenoic acid, 1-glutamic acid, heptanoic acid, hexanoic acid, 3-hexenoic acid, trans-2-hexenoic acid, isobutyric acid, lauric acid, 2-methylbutyric acid, 2-methylvaleric acid, myristic acid, nonanoic acid, palmitic acid, 4-pentenoic acid, phenylacetic acid, 3-phenylpropionic acid, phosphoric acid and sulfuric acid.

82. The liquid aerosol formulation of claim 73, wherein the acidic compound comprises equal parts of each of the pyruvic acid, the lactic acid, the benzoic acid and the acetic acid.

83. The liquid aerosol formulation of claim 72, wherein a concentration of the nicotine is substantially 3% by weight.

84. The liquid aerosol formulation of claim 83, wherein a concentration of pyruvic acid is substantially 0.5%, a concentration of lactic acid is substantially 0.4%, and a concentration of benzoic acid is substantially 0.1%.

85. The liquid aerosol formulation of claim 83, wherein a concentration of pyruvic acid is substantially 0.5%, a concentration of lactic acid is substantially 0.4%, and a concentration of hydrochloric acid is substantially 0.1%.

86. The liquid aerosol formulation of claim 83, further comprising substantially 0.5% nicotine bitartrate and wherein a concentration of hydrochloric acid is substantially 0.2%.

87. The liquid aerosol formulation of claim 83, wherein a concentration of pyruvic acid is substantially 0.5%, a concentration of lactic acid is substantially 0.4%, and a concentration of tartaric acid is substantially 0.5%.

88. The liquid aerosol formulation of claim 83, wherein a concentration of pyruvic acid is substantially 0.5%, a concentration of lactic acid is substantially 0.4%, a concentration of benzoic acid is substantially 0.1% and a concentration of oleic acid is substantially 0.1%.

89. The liquid aerosol formulation of claim 83, wherein a concentration of nicotine bitartrate is substantially 0.5%, a concentration of pyruvic acid is substantially 0.5%, a concentration of lactic acid is substantially 0.4% and a concentration of benzoic acid is substantially 0.1%.

90. The liquid aerosol formulation of claim 83, wherein a concentration of nicotine bitartrate is substantially 0.5%, a concentration of pyruvic acid is substantially 0.5%, a concentration of lactic acid is substantially 0.4%, a concentration of benzoic acid is substantially 0.1% and a concentration of oleic acid is substantially 1%.

91. The liquid aerosol formulation of claim 72, wherein a concentration of the nicotine is substantially 3% to 5% by weight.

92. The liquid aerosol formulation of claim 91, wherein a concentration of formic acid is substantially 0.036% to 0.144%, a concentration of pyruvic acid is substantially 0.35% to 1.4%, a concentration of oxalic acid is substantially 0.013% to 0.052%, and a concentration of glycolic acid is substantially 0.05% to 0.2%.

93. The liquid aerosol formulation of claim 92, wherein the concentration of nicotine is substantially 3% by weight, the concentration of formic acid is substantially 0.036%, the concentration of pyruvic acid is substantially 0.35%, the concentration of oxalic acid is substantially 0.013% and the concentration of glycolic acid is substantially 0.05%.

94. The liquid aerosol formulation of claim 91, further comprising substantially 1% to 2% nicotine bitartrate and wherein a concentration of acetic acid is substantially 0.4% to 0.9%.

95. The liquid aerosol formulation of claim 94, wherein the concentration of nicotine is substantially 3% by weight, the concentration of nicotine bitartrate is substantially 1% and the concentration of acetic acid is substantially 0.4%.

96. The liquid aerosol formulation of claim 91, further comprising substantially 0.5% to 1% nicotine bitartrate and wherein a concentration of a mixture of substantially equal parts pyruvic acid, benzoic acid, lactic acid and acetic acid is substantially 0.5% to 1%.

97. The liquid aerosol formulation of claim 91, further comprising substantially 0.5% to 1% nicotine bitartrate and wherein a concentration of hydrochloric acid is substantially 0.01% to 0.1%.

98. The liquid aerosol formulation of claim 91, wherein a concentration of oleic acid is substantially 0.5% to 1% and a concentration of a mixture of substantially equal parts pyruvic acid, benzoic acid, lactic acid and acetic acid is substantially 0.5% to 1.5.

99. The liquid aerosol formulation of claim 91, wherein a concentration of sorbic acid is substantially 0.1% to 0.5%, a concentration of tartaric acid is substantially 0.1% to 0.5%, and a concentration of pyruvic acid is substantially 0.1% to 0.75%.

100. The liquid aerosol formulation of claim 99, further comprising substantially 0.01% to 0.1% hydrochloric acid, wherein the concentration of nicotine is substantially 5% by weight.

101. The liquid aerosol formulation of claim 91, wherein a concentration of succinic acid is substantially 0.1% to 0.5%, a concentration of pyruvic acid is substantially 0.1% to 0.75%, and a concentration of lactic acid is substantially 0.1% to 0.5%.

102. The liquid aerosol formulation of claim 101, further comprising at least one of substantially 0.01% to 0.1% hydrochloric acid and substantially 0.1% to 0.25% tartaric acid, wherein the concentration of nicotine is substantially 5% by weight.

103. The liquid aerosol formulation of claim 81, wherein an amount of char produced via the acidic compound is substantially lower than an amount of char produced via another acidic compound.

104. The liquid aerosol formulation of claim 73, wherein the acidic compound comprises more of the pyruvic acid than one or more of the lactic acid, the benzoic acid and the acetic acid.

105. The liquid aerosol formulation of claim 73, wherein the acidic compound comprises substantially 20% to 30% of each of the pyruvic acid, the lactic acid, the benzoic acid and the acetic acid.

106. The liquid aerosol formulation of claim 73, wherein the acidic compound comprises substantially 15% to 35% of each of the pyruvic acid, the lactic acid, the benzoic acid and the acetic acid.

107. The liquid aerosol formulation of claim 73, wherein the acidic compound comprises substantially 10% to 40% of each of the pyruvic acid, the lactic acid, the benzoic acid and the acetic acid.

108. The liquid aerosol formulation of claim 73, wherein the acidic compound comprises substantially 5% to 45% of each of the pyruvic acid, the lactic acid, the benzoic acid and the acetic acid.

109. The liquid aerosol formulation of claim 73, wherein the acidic compound comprises less than substantially 50% of each of the pyruvic acid, the lactic acid, the benzoic acid and the acetic acid.
